(19) United States
(12) Patent Application Publication
(10) Pub. No.: US 2015/0305903 A1
(43) Pub. Date: Oct. 29, 2015

(54) SELF-EXPANDABLE STENT SYSTEM

(71) Applicant: TERUMO KABUSHIKI KAISHA, Tokyo (JP)

(72) Inventor: Takashi KITAOKA, Hadano-shi (JP)

(73) Assignee: TERUMO KABUSHIKI KAISHA, Tokyo (JP)

(21) Appl. No.: 14/796,660

(22) Filed: Jul. 10, 2015

Related U.S. Application Data
(63) Continuation of application No. PCT/JP2013/051334, filed on Jan. 23, 2013.

(51) Int. Cl. A61F 2/966; A61F 2/958

(52) U.S. Cl. CPC A61F 2/966; A61F 2/958; A61F 2230/0069

(57) ABSTRACT

A self-expandable stent system is disclosed, which includes an inner structure and an outer tube, and a self-expandable stent, which is arranged between the inner structure and the outer tube. A distal end dilating portion is disposed in a distal end of the inner structure, and the distal end dilating portion closes an opening disposed in a distal end of the outer tube.

### CROSS-REFERENCES TO RELATED APPLICATIONS

This application is a continuation of International Application No. PCT/JP2013/051334 filed on Jan. 23, 2013, the entire content of which is incorporated herein by reference.

### TECHNICAL FIELD

The present disclosure generally relates to a self-expandable stent system having an inner structure and an outer tube.

### BACKGROUND DISCUSSION

As a medical device to be inserted into a body, a stent delivery system having a stent can be used in treating myocardinal infarction or angina pectoris. For example, the stent delivery system is inserted into the body and the stent is caused to expand in a lesion (stenosis) of a coronary artery. In this manner, a lumen in the coronary artery can be secured by spreading out the stenosis.

A type of the above-described stent includes a self-expandable stent, which can expand by itself. A stent delivery system having this self-expandable stent has been known (refer to Japanese Patent No. 4733055).

The stent delivery system disclosed in Japanese Patent No. 4733055 has a distal tip including a scallop-processed surface, a sheath which is in close contact with the distal tip and surrounds a catheter, and a stent which is arranged in a space between the catheter and the sheath. The stent delivery system employs a structure where a gap is formed between the scallop-processed surface of the distal tip and a distal edge of the sheath.

In this case, as illustrated by an arrow in FIG. 7C in Japanese Patent No. 4733055, when a cleaning solution is caused to flow into the space between the catheter and the sheath, the cleaning solution passes through the gap and flows outward from the sheath. Accordingly, a so-called priming can be performed in which the cleaning solution pushes air inside the sheath outward through the gap in a state where the distal edge of the sheath and the distal tip are brought into close contact with each other.

However, a size of the gap formed between the scallop-processed surface of the distal tip and the distal edge of the sheath can be very small as compared to an inner diameter of the sheath. Consequently, a discharge amount of the air discharged from the gap is limited, and the air is less likely to be removed during the priming, thereby causing a disadvantage in that a desired object may not be achieved. In addition, since the gap is present, when a distal portion of the stent delivery system is inserted into the body, there is a possibility that the blood may flow backward into the sheath. Further, when a device such as the stent indwells in the body in advance and the distal portion of the stent delivery system passes through the inside of the device, the gap is caught on an end portion of the device, thereby causing a possibility that the end portion of the device may enter the gap.

Furthermore, the distal edge of the sheath can be exposed in a portion having the gap. Consequently, a step difference occurs between a head portion of the distal tip and the distal edge of the sheath, thereby causing a disadvantage in that the distal edge of the sheath hits against a vascular wall.

### SUMMARY

A self-expandable stent system is disclosed, which can efficiently and sufficiently discharge air outward from a gap formed between an outer tube and an inner tube (inner structure) both of which configure double tubes such as a sheath and a catheter during priming, and which can be easily and reliably inserted into a lumen without a distal edge of the outer tube hitting against a lumen inner wall of a blood vessel.

A self-expandable stent system is disclosed, which can include an inner structure, an outer tube, and a self-expandable stent arranged between the inner structure and the outer tube. A distal end of the inner structure has a distal end dilating portion, which comes into close contact with a distal end of the outer tube when a contraction state is switched over to a dilation state.

In accordance with an exemplary embodiment, before the self-expandable stent system is used, the distal end dilating portion is in a contraction state, and an opening disposed in a distal end of the outer tube is in an opened state. In this state, when priming fluid is caused to flow from a proximal side of the system to a gap formed between the outer tube and the inner structure, the fluid pushes air present in the gap outward, thereby enabling the air inside the gap to be efficiently discharged outward. In addition, before the system is inserted into a body, the distal end dilating portion is brought into the dilation state. In this manner, the opening disposed in the distal end of the outer tube is closed, and a step difference does not occur between the distal end dilating portion and a distal edge of the outer tube. Accordingly, the system can be inserted into a lumen without the distal edge of the outer tube hitting against a lumen inner wall.

In accordance with an exemplary embodiment, the distal end dilating portion may be formed from a balloon which dilates by being pressurized, or may be formed of a molded material of swellable gel which swells by absorbing liquid.

In accordance with an exemplary embodiment, the distal end dilating portion can contract so as to have a decreased size before the system is used, thereby opening the distal end of the outer tube. Accordingly, efficient priming can be realized. In addition, the distal end dilating portion is caused to inflate (dilate or swell) before the system is used. Accordingly, a step difference does not occur between the distal end dilating portion and the distal edge of the outer tube.

In accordance with an exemplary embodiment, efficient priming can be realized by disposing a large opening in the distal end of the outer tube. In addition, the distal end dilating portion closes the opening and can help eliminate the step difference with the outer tube after the priming is completed. Therefore, it becomes relatively easy to insert the system into the lumen.

### BRIEF DESCRIPTION OF THE DRAWINGS

FIG. 1 is a partially omitted overall explanatory view for illustrating a self-expandable stent system according to a first embodiment of the present disclosure.

FIG. 2 is a partially omitted enlarged vertical cross-sectional view of a distal portion of the self-expandable stent system in FIG. 1.

FIG. 3 is a cross-sectional view taken along line III-III in FIG. 2.

FIG. 4A is a partially omitted vertical cross-sectional view for illustrating a flow of fluid during priming in the self-expandable stent system illustrated in FIG. 1.

FIG. 4B is a partially omitted enlarged cross-sectional view for illustrating a flow of balloon pressurizing fluid in the self-expandable stent system illustrated in FIG. 1.

FIG. 4C is a partially omitted vertical cross-sectional view for illustrating a state where a balloon further dilates from a state illustrated in FIG. 4B.

FIG. 5 is a partially omitted vertical cross-sectional view illustrating a modification example of the distal portion of the self-expandable stent system illustrated in FIG. 1.

FIG. 6A is an enlarged vertical cross-sectional view of a distal portion of a self-expandable stent system according to a second embodiment of the present disclosure.

FIG. 6B is an enlarged vertical cross-sectional view for illustrating a state where a molded material of swellable gel swells in the self-expandable stent system illustrated in FIG. 6A.

FIG. 7A is an enlarged vertical cross-sectional view of a distal portion of a self-expandable stent system according to a third embodiment of the present disclosure.

FIG. 7B is an enlarged vertical cross-sectional view for illustrating a state where a balloon dilates in the self-expandable stent system illustrated in FIG. 7A.

FIG. 8A is an enlarged vertical cross-sectional view of a distal portion of a self-expandable stent system according to a fourth embodiment of the present disclosure.

FIG. 8B is an enlarged vertical cross-sectional view for illustrating a state where a molded material of swellable gel swells in the self-expandable stent system illustrated in FIG. 8A.

### DETAILED DESCRIPTION

Hereinafter, a self-expandable stent system according to the present disclosure will be described in detail with reference to exemplary embodiments and the accompanying drawings.

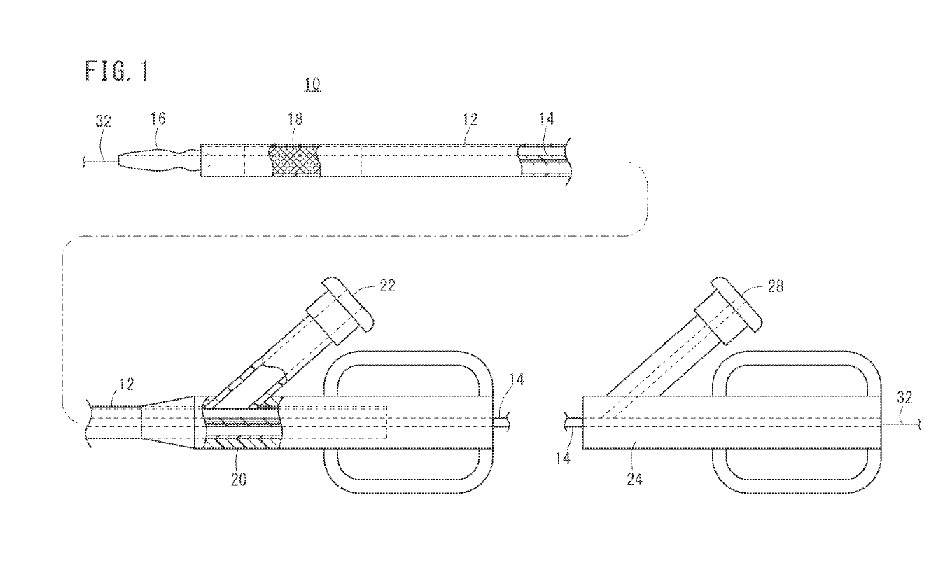

As illustrated in FIG. 1, a self-expandable stent system 10 according to a first embodiment has an elongated and hollow sheath (outer tube) 12 and a hollow and small diameter shaft (inner structure) 14 which is inserted into the sheath 12 and which can relatively move to and from the sheath 12 in an axial direction. Here, the self-expandable stent system 10 is in an unsealed state after a medical packing is unpacked.

For example, a balloon-type distal tip (distal end dilating portion) 16 which dilates by pressure generated when a contrast medium is injected to serve as balloon pressurizing fluid is disposed in the distal portion of the shaft 14. The balloon-type distal tip 16 is in a contraction state before use, as in a state before the medical package is unpacked. The balloon-type distal tip 16 dilates before being inserted into a body. Hereinafter, description will be made on the assumption that the balloon-type distal tip 16 side is the distal side of the self-expandable stent system 10 and a second hub 24 side (to be described later) is the proximal side of the self-expandable stent system 10.

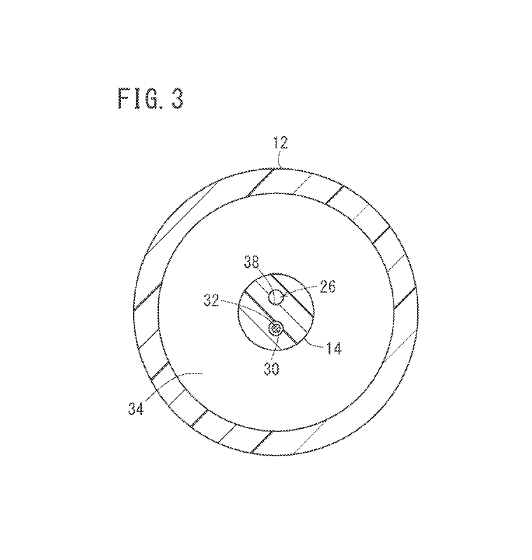

A self-expandable stent 18 which can expand by itself is arranged in a portion (gap 34 to be described later) between the shaft 14 and the sheath 12. A first hub 20 extending in the axial direction is fixed to the proximal side of the sheath 12, and a first fluid injection port 22 communicating with a space, for example, a gap formed between the sheath 12 and the shaft 14 is disposed integrally with the first hub 20. As will be described later, the shaft 14 internally has a balloon pressurizing lumen 26 and a guidewire lumen 30 (refer to FIG. 3).

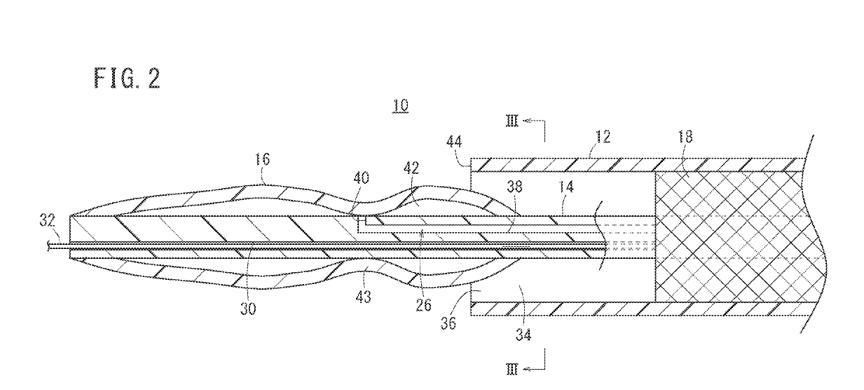

An intermediate portion of the shaft 14 is inserted into the first hub 20 so as to be movable along the axial direction. In contrast, in accordance with an exemplary embodiment, the proximal portion of the shaft 14 can be fixed to the second hub 24. A second fluid injection port 28 communicating with the balloon pressurizing lumen 26 (refer to FIGS. 2 and 3) inside the shaft 14 can be integrally disposed in the second hub 24. Furthermore, a guidewire 32 can be inserted into the guidewire lumen 30 inside the shaft 14 over the total length of the shaft 14. As illustrated in FIG. 3, the shaft 14 has a double lumen structure including the balloon pressurizing lumen 26 and the guidewire lumen 30.

As illustrated in FIG. 2, a gap 34 communicating with the first fluid injection port 22 is formed between the sheath 12 and the shaft 14 (refer to FIG. 2). In the drawing, the reference numeral 36 represents an opening formed in the distal end of the sheath 12.

The balloon pressurizing lumen 26 is configured to include an axial hole portion 38 which extends in the axial direction of the shaft 14, and a radial hole portion 40 which communicates with the axial hole portion 38 and extends outward in the radial direction of the shaft 14.

Next, the balloon-type distal tip 16 will be described. As shown in FIG. 2, the balloon-type distal tip 16 can be a flexible, dilatable, and contractible sack-like body, which extends along the axial direction of the balloon pressurizing lumen 26. The distal portion of the tip 16 can be fixedly attached so as to match the distal portion of the shaft 14. The proximal portion of the tip 16 extends beyond a distal edge 44 of the sheath 12 to the inside of the sheath 12, and is fixedly attached to a peripheral wall of the shaft 14 on the proximal side relative to the distal edge 44. The proximal portion of the balloon-type distal tip 16 may be fixedly attached to the peripheral wall of the shaft 14, which is located at the same position as the distal edge 44. The balloon-type distal tip 16 can include a decreased diameter portion 43 in the vicinity of the portion where a space 42 is formed inside the balloon-type distal tip 16. FIG. 2 illustrates a state where an inner wall of the decreased diameter portion 43 is in contact with the radial hole portion 40 of the shaft 14. A position of the decreased diameter portion 43 is not bound to a portion of the radial hole portion 40. In accordance with an exemplary embodiment, any configuration may be adopted as long as the balloon-type distal tip 16 is in a contraction state before use. For example, the balloon-type distal tip 16 may be configured to include materials of nylon, nylon elastomer, polyester such as polyethylene terephthalate, and the like, polyester elastomer, polyolefin such as polyethylene, polypropylene, and the like, polyurethane, a silicone rubber, or the like.

Next, an operation and an advantageous effect of the self-expandable stent system 10 according to the first embodiment configured as described above will be described.

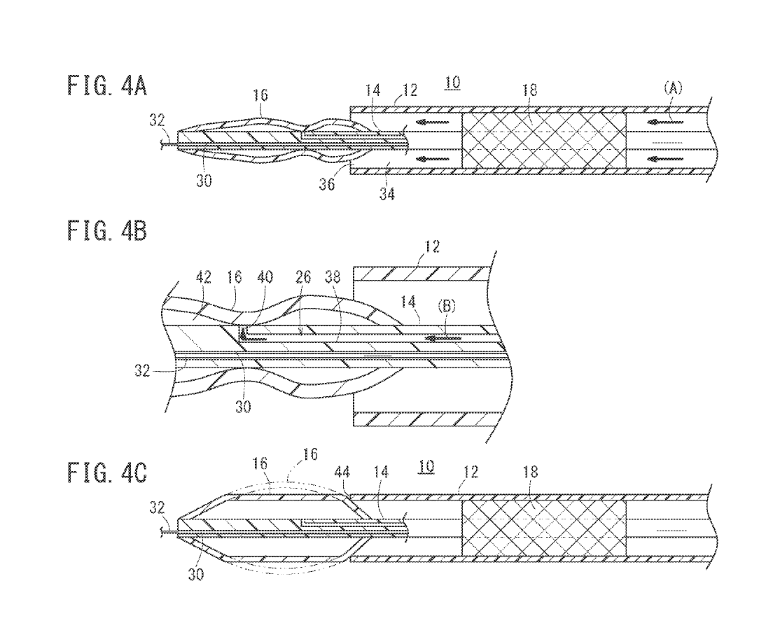

As illustrated in FIG. 4A, the balloon-type distal tip 16 after the medical package is unpacked is in a contraction state. The balloon-type distal tip 16 and the distal end of the sheath 12 are not in close contact with each other. Accordingly, the distal end of the sheath 12 is in an opened state. In this state, when a saline solution is caused to flow into the gap 34 formed between the sheath 12 and the shaft 14 through the first fluid injection port 22 located on the proximal side of the self-expandable stent system 10, the saline solution pushes air present in the gap 34 outward as illustrated by an arrow (A). Accordingly, the air inside the gap 34 can be efficiently discharged outward (for example, efficient priming can be realized).

Next, as illustrated in FIG. 4B, liquid such as a contrast medium and the like is injected through the second fluid injection port 28 (refer to FIG. 1). The injected liquid passes through the axial hole portion 38 and the radial hole portion 40 of the balloon pressurizing lumen 26 as illustrated by an arrow (B), and flows into the space 42 inside the balloon-type distal tip 16.

As a result, as illustrated in FIG. 4C, the balloon-type distal tip 16 including the decreased diameter portion 43 expands and dilates by pressure of the liquid flowing into the gap 42. The proximal side of the balloon-type distal tip 16 comes into close contact with an inner peripheral end portion of the distal edge 44 of the sheath 12 and closes an opening 36 of the sheath 12. Subsequently, the balloon-type distal tip 16 and the distal edge 44 of the sheath 12 can be integrated with each other. When the balloon-type distal tip 16 and the distal edge 44 of the sheath 12 are integrated with each other in this way, a step difference no longer occurs in the radial direction between both of these as illustrated in FIG. 4C. Accordingly, when the self-expandable stent system 10 is inserted into the blood vessel in the body, the self-expandable stent system 10 can be inserted without the distal edge 44 of the sheath 12 hitting against a lumen inner wall such as a vascular inner wall and the like.

Moreover, the opening 36 is closed by the balloon-type distal tip 16 and the distal edge 44 of the sheath 12 coming into close contact with each other. As a result, when the self-expandable stent system 10 is inserted into the body, blood can be prevented from flowing backward into the sheath 12.

As illustrated by a two-dot chain line in FIG. 4C, the balloon-type distal tip 16 may be caused to dilate further outward beyond an outer diameter dimension of the sheath 12, which can more reliably prevent blood from flowing backward into the sheath 12.

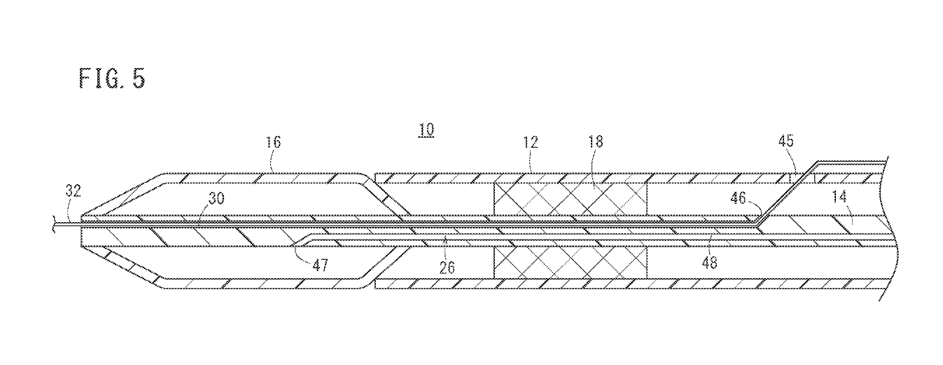

In accordance with an exemplary embodiment, the present embodiment employs an over-the-wire structure (OTW structure) in which the guidewire lumen 30 extends to the second hub 24. However, the present embodiment may employ a rapid exchange structure (RX structure) in which the opening 36 is disposed in the middle of the sheath 12 and the proximal end of the guidewire lumen 30 of the shaft 14 is caused to communicate with the outside of the shaft 14 via the opening 36 of the sheath 12. FIG. 5 illustrates a modification example of the distal portion of the self-expandable stent system, which employs the RX structure.

In this modification example, the same reference numerals are given to configuration elements which are the same as those in the first embodiment, and detailed description thereof will be omitted.

Therefore, in this modification example, an opening 45 is formed in the middle of the sheath 12. In contrast, an opening 46, which has a smaller diameter than the opening 45, is formed in the middle of the shaft 14. Furthermore, an opening 47 facing the inside of the balloon-type distal tip 16 is formed on the distal side of the shaft 14, and an axial hole portion 48 communicating with the opening 47 is disposed thereon.

In this modification example, the guidewire 32 is inserted from the opening 45, is passed through the opening 46, and is exposed outward from the distal portion of the shaft 14 through the guidewire lumen 30. In contrast, the axial hole portion 48 and the opening 47 are used in injecting liquid such as a contrast medium and the like therethrough.

According to this modification example, an operation effect, which is the same as that in the first embodiment can also be obtained.

Incidentally, the self-expandable stent system 10 employs the balloon-type distal tip 16. In accordance with an exemplary embodiment, a distal tip is disclosed for achieving an operation effect, which is the same as that of the balloon-type distal tip 16 by employing a molded material of swellable gel, which can swell by absorbing fluid, particularly liquid. Next, the above configuration will be described as a second embodiment.

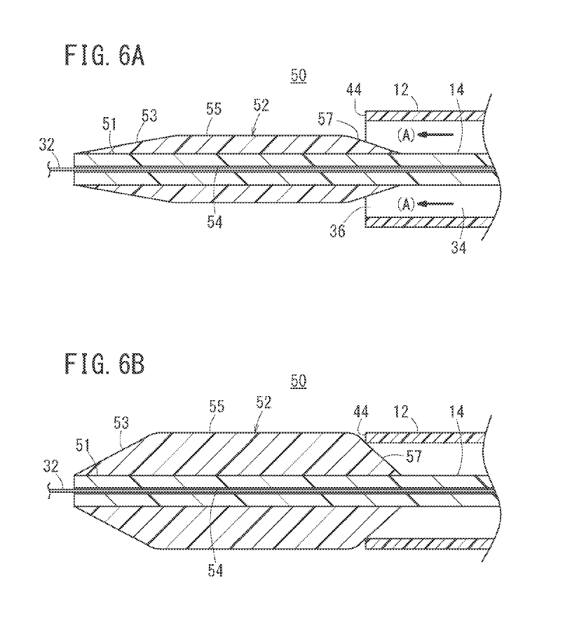

A self-expandable stent system 50 according to the second embodiment will be described with reference to FIG. 6A. In accordance with an exemplary embodiment, a the balloon-type distal tip 16 employed in the first embodiment is changed to a swellable distal tip. Therefore, the same reference numerals are given to configuration elements, which are the same as the configuration elements in FIGS. 1 to 5 used to describe the first embodiment. Detailed description thereof will be omitted, and the rest is the same as above.

As illustrated in FIG. 6A, the self-expandable stent system (medical device) 50 according to the second embodiment can include a swellable distal tip (distal end dilating portion) 52, which can be fixed to the distal portion of the shaft 14 and which is formed of a molded material of swellable gel. The swellable distal tip 52 has a through-hole 51 extending in the axial direction. The shaft 14 is inserted into the through-hole 51, and an outer peripheral wall of the shaft 14 is in close contact with an inner peripheral wall of the through-hole 51. A first tapered portion 53 is formed in the distal portion of the swellable distal tip 52, and a second tapered portion 57 is formed on the proximal side through a body portion 55. The shaft 14 can include a guidewire lumen 54 extending in the axial direction. In accordance with an exemplary embodiment, the guidewire 32 can be freely inserted into the guidewire lumen 54. For example, as a material used for the molded material of swellable gel, it can be preferable to use polyvinyl alcohol, polyethylene glycol, sodium polyacrylate, or the like.

The self-expandable stent system 50 according to the second embodiment is configured as described above. Next, an operation effect of the self-expandable stent system 50 will be described.

In the self-expandable stent system 50, when a saline solution is caused to flow into the gap 34 during priming as illustrated by an arrow (A), the saline solution comes into contact with the swellable distal tip 52, and the swellable distal tip 52 starts to swell by absorbing the saline solution. As a result, as illustrated in FIG. 6B, the second tapered portion 57 on the proximal side of the swellable distal tip 52 comes into close contact with an inner peripheral edge of the distal edge 44 of the sheath 12, thereby closing the opening 36. If necessary, the second tapered portion 57 of the swellable distal tip 52 may have a shape of swelling further outward beyond the outer diameter dimension of the sheath 12.

As illustrated in FIG. 6A, the swellable distal tip 52 is in a contraction state in an environment where the swellable distal tip 52 is dried after the medical package is unpacked. Therefore, the opening 36 of the sheath 12 is opened. Therefore, the air pushed outward by the saline solution passing therethrough can be reliably discharged from the opening 36. Accordingly, efficient priming can be realized. In addition, before the self-expandable stent system 50 is inserted into the body, the swellable distal tip 52 swells by absorbing moisture of the saline solution used in the priming as illustrated in FIG. 6B. In accordance with an exemplary embodiment, an integrated structure can be realized having no step difference between the swellable distal tip 52 and the distal edge 44 of the sheath 12. Furthermore, in order to cause the swellable distal tip 52 to dilate, only the saline solution for priming may be caused to flow into the swellable distal tip 52, and it is not necessary to separately inject distal tip dilating liquid, and the burden to an operator can be relatively eased.

In the self-expandable stent system 50 described with reference to FIGS. 6A and 6B, the overall swellable distal tip 52 can be switched from a contraction state to a dilation state. However, in order to facilitate the priming by sufficiently discharging the air inside the sheath 12 and to obtain an integral structure having no step difference between the distal edge 44 of the sheath 12 and the swellable distal tip 52, the present disclosure is not limited to the above-described embodiments. For example, only a portion coming into close contact with the distal edge 44 of the sheath 12 within the balloon-type distal tip 16 may be switched from the contraction state to the dilation state. Next, a corresponding embodiment will be described.

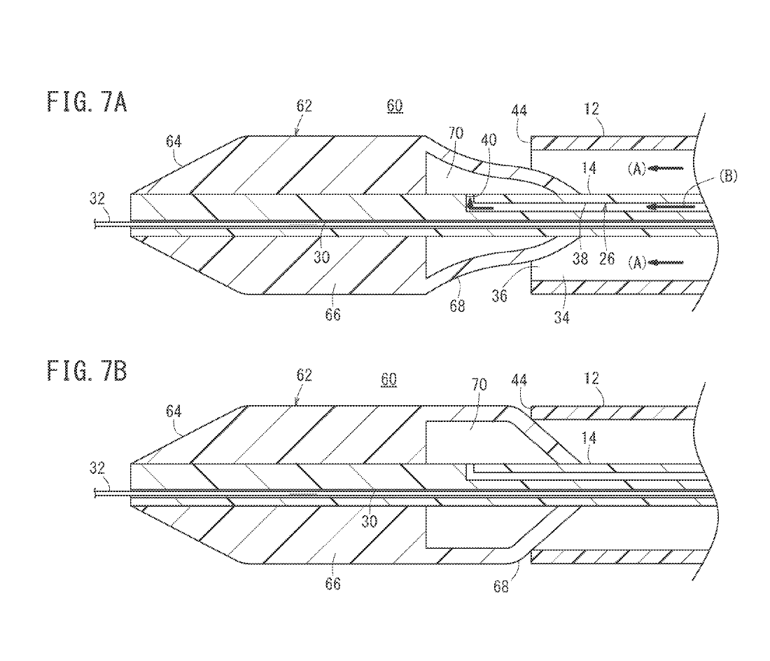

A self-expandable stent system 60 according to a third embodiment in which a shape of the distal tip is changed in the self-expandable stent system 10 according to the first embodiment will be described with reference to FIGS. 7A and 7B. In accordance with an exemplary embodiment, a distal tip 62 is disposed in the distal portion of the shaft 14, but a shape of the distal tip 62 is different from that in the first embodiment.

In accordance with an exemplary embodiment, in the self-expandable stent system 60 according to the third embodiment, the distal tip 62 has a tapered portion 64 whose distal side has an acuminate shape, and can include a balloon portion (distal end dilating portion) 68 in which a middle portion in the axial direction forms a body portion 66 and which extends from the body portion 66 to the proximal side. The proximal portion of the balloon portion 68 can extend from the distal edge 44 of the sheath 12 to the inside of the sheath 12, and is fixedly attached to a peripheral wall of the shaft 14 on the proximal side relative to the distal edge 44. The proximal portion of the balloon portion 68 may be fixedly attached to the peripheral wall of the shaft 14 located at the same position as the distal edge 44. A space portion 70 is formed inside the balloon portion 68. The space portion 70 communicates with the radial hole portion 40. For example, the balloon portion 68 may be configured to include materials of nylon, nylon elastomer, polyester such as polyethylene terephthalate, and the like, polyester elastomer, polyolefin such as polyethylene, polypropylene, and the like, polyurethane, a silicone rubber, or the like.

In the above-described configuration, the saline solution is caused to flow into the gap 34 between the sheath 12 and the shaft 14 through the first fluid injection port 22, and the air inside the gap 34 is discharged outward from the opening 36, thereby performing the priming. Thereafter, as illustrated by an arrow (B), liquid such as a contrast medium and the like is injected through the second fluid injection port 28, passes through the axial hole portion 38 and the radial hole portion 40 of the balloon pressurizing lumen 26, and flows into the space portion 70 inside the balloon portion 68.

As a result, as illustrated in FIG. 7B, the space portion 70 of the balloon portion 68 is caused to dilate by the pressure of the liquid. The proximal side of the balloon portion 68 comes to have a tapered shape, and comes into close contact with an inner peripheral edge of the distal edge 44 of the sheath 12. As compared with the balloon-type distal tip 16 (refer to FIG. 2) according to the first embodiment, the balloon portion 68 has a smaller volume of the space portion 70 inside the balloon portion 68. Accordingly, an amount of the liquid to be injected can be minimized. When the balloon portion 68 dilates by injecting the liquid, the balloon portion 68 may swell outward beyond the outer diameter dimension of the distal edge 44 of the sheath 12. In accordance with an exemplary embodiment, any configuration may be adopted as long as adhesion can be ensured between the distal edge 44 and the balloon portion 68.

In the self-expandable stent system 50 (refer to FIGS. 6A and 6B) described according to the second embodiment, the overall swellable distal tip 52 can be switched from a contraction state to a dilation state. In addition, in the self-expandable stent system 60 (refer to FIGS. 7A and 7B) described according to the third embodiment, only the balloon portion 68 disposed on the proximal side of the balloon-type distal tip 62 is switched to the dilation state. Based on this consideration, only a proximal side portion coming into close contact with the distal edge 44 of the sheath 12 within the swellable distal tip may be switched from the contraction state to the dilation state. Next, the above configuration will be described as a fourth embodiment.

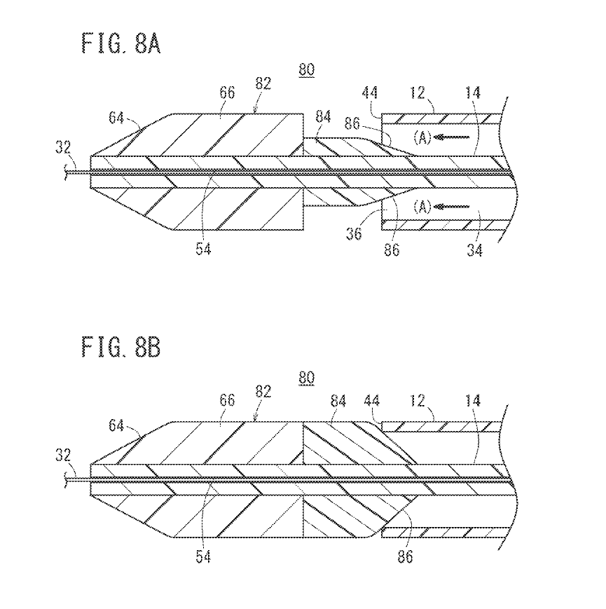

FIGS. 8A and 8B illustrate a self-expandable stent system 80 according to the fourth embodiment. In accordance with an exemplary embodiment, as compared to the third embodiment illustrated in FIGS. 7A and 7B, the fourth embodiment includes a swelling portion 84 which is a molded material of swellable gel disposed on the proximal side of a distal tip 82.

In accordance with an exemplary embodiment, for example, the shape of the distal tip 82 is the same as that of the tapered portion 64 and the body portion 66 which are described according to the third embodiment. However, this embodiment is different from the third embodiment in that the swelling portion 84 extending to the proximal side is disposed in a terminal end portion of the body portion 66. The swelling portion 84 can include a tapered portion 86 which extends from the distal edge 44 of the sheath 12 to the inside of the sheath 12 and which is fixedly attached to the outer peripheral surface of the shaft 14. In accordance with an exemplary embodiment, the proximal end of the swelling portion 84 may serve as the distal edge 44.

For example, the tapered portion 64 and the body portion 66 which are integrated with each other may be configured to include materials of nylon, nylon elastomer, polyester such as polyethylene terephthalate, and the like, polyester elastomer, polyolefin such as polyethylene, polypropylene, and the like, polyurethane, a silicone rubber, or the like. In addition, for example, the swelling portion 84 may be configured to include materials of polyvinyl alcohol, polyethylene glycol, sodium polyacrylate, or the like.

In the self-expandable stent system 80 described according to the fourth embodiment configured as described above, when the saline solution is caused to flow into the gap 34 during the priming as illustrated by an arrow (A), the saline solution discharges the air outward via the gap 34, and a part of the saline solution partially comes into contact with the swelling portion 84. In this manner, the swelling portion 84 starts to swell by absorbing the saline solution. As a result, as illustrated in FIG. 8B, the tapered portion 86 of the swelling portion 84 can come into close contact with the inner peripheral edge of the distal edge 44 of the sheath 12, thereby closing the opening 36. As compared to the swellable distal tip 52 (refer to FIGS. 6A and 6B) according to the second embodiment, the swelling portion 84 has fewer portions formed of the molded material of the swellable gel. Accordingly, the swelling portion 84 can quickly swell using a small amount of the saline solution.

The swelling portion 84 is the same as those in the other embodiments in that the swelling portion 84 may swell outward beyond the outer diameter dimension of the sheath 12.

The detailed description above describes a self-expandable stent system. The invention is not limited, however, to the precise embodiments and variations described. Various changes, modifications and equivalents can effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A self-expandable stent system comprising:
an inner structure;
an outer tube; and
a self-expandable stent arranged between the inner structure and the outer tube, wherein
a distal end of the inner structure has a distal end dilating portion which comes into close contact with a distal end of the outer tube when a contraction state is switched over to a dilation state.

2. The self-expandable stent system according to claim 1, wherein
the distal end dilating portion is formed from a balloon which dilates by being pressurized.

3. The self-expandable stent system according to claim 1, wherein
the distal end dilating portion is formed of a molded material of swellable gel which swells by absorbing liquid.

4. The self-expandable stent system according to claim 1, wherein
a distal portion of the inner structure protrudes outward beyond the outer tube,
a distal portion of the distal end dilating portion is fixedly attached to the distal portion of the inner structure, and
a proximal portion of the distal end dilating portion is fixedly attached to the inner structure which is arranged at the same position as a distal edge of the outer tube, or on a proximal side relative to the distal edge.

5. The self-expandable stent system according to claim 2, wherein
the inner structure has a guidewire lumen which extends in an axial direction and a balloon pressurizing lumen for introducing fluid into the distal end dilating portion.

6. The self-expandable stent system according to claim 1, wherein
an opening is disposed in the distal end of the outer tube, and
after priming fluid is caused to flow via the opening the opening is closed.

7. The self-expandable stent system according to claim 6, wherein
the opening is closed by dilation of the distal end dilating portion of a balloon type.

8. The self-expandable stent system according to claim 7, wherein
when the opening is closed, there is no substantial step difference between a dilated portion of the distal end dilating portion and an outer peripheral surface of the outer tube.

9. The self-expandable stent system according to claim 6, wherein
the opening is closed by swelling of the distal end dilating portion of a swellable type.

10. The self-expandable stent system according to claim 9, wherein
when the opening is closed, there is no substantial step difference between a swelled portion of the distal end dilating portion and an outer peripheral surface of the outer tube.

11. The self-expandable stent system according to claim 1, wherein
the distal end dilating portion has a tapered shape whose distal side is acuminate.

12. The self-expandable stent system according to claim 1, wherein
the distal end dilating portion has a tapered shape whose proximal side is acuminate.

13. The self-expandable stent system according to claim 2, wherein
the distal end dilating portion is formed of any one material of nylon, nylon elastomer, polyester, polyester elastomer, polyolefin, polyurethane, and silicone rubber.

14. The self-expandable stent system according to claim 3, wherein
the distal end dilating portion is formed of any one material of polyvinyl alcohol, polyethylene glycol, and sodium polyacrylate.

15. The self-expandable stent system according to claim 2, wherein
a distal portion of the inner structure protrudes outward beyond the outer tube,
a distal portion of the distal end dilating portion is fixedly attached to the distal portion of the inner structure, and
a proximal portion of the distal end dilating portion is fixedly attached to the inner structure which is arranged at the same position as a distal edge of the outer tube, or on a proximal side relative to the distal edge.

16. The self-expandable stent system according to claim 3, wherein
a distal portion of the inner structure protrudes outward beyond the outer tube,
a distal portion of the distal end dilating portion is fixedly attached to the distal portion of the inner structure, and
a proximal portion of the distal end dilating portion is fixedly attached to the inner structure which is arranged at the same position as a distal edge of the outer tube, or on a proximal side relative to the distal edge.
